(19) United States
(12) Patent Application Publication
(10) Pub. No.: US 2014/0339337 A1
(43) Pub. Date: Nov. 20, 2014

(54) AUTOMATIC AROMA SPRAYER

(71) Applicant: SERENE HOUSE INTERNATIONAL ENTERPRISE LTD., Road Town (VG)

(72) Inventor: Ming Jen HSIAO, Miaoli County (TW)

(73) Assignee: SERENE HOUSE INTERNATIONAL ENTERPRISE LTD., Road Town (VG)

(21) Appl. No.: 13/893,448

(22) Filed: May 14, 2013

(51) Int. Cl. A61L 9/14; B05B 9/043

(52) U.S. Cl. CPC A61L 9/14; B05B 9/043
USPC 239/332

(57) ABSTRACT

An automatic aroma sprayer formed of a housing, a bottom cap, a lower gearbox shell, an upper gearbox shell, a pressure bar, a motor, a sprayer head, a functional circuit board and a power supply unit is disclosed. The functional circuit board controls the motor to rotate a gear set in moving a pressure bar inside the upper gearbox shell to press a top nozzle tip of an aroma bottle, causing the sprayer head to spray a predetermined amount of a fine spray of liquefied aromatic substance out of a glass aroma bottle automatically at a predetermined time interval.

### BACKGROUND OF THE INVENTION

1. Field of the Invention

The present invention relates to nebulizers and more particularly, to an automatic aroma sprayer, which automatically sprays a predetermined amount of fine mist of liquefied aromatic substance at a predetermined time interval.

2. Description of the Related Art

Various designs of aromatic nebulizing diffusers and aroma sprayers are commercially available. An aromatic nebulizing diffuser generally comprises an enclosed container holding an essential oil or liquefied aromatic substance that can be heated into vapor and diffused into the outside open air when the aromatic nebulizing diffuser is turned on. However, when the aromatic nebulizing diffuser is turned on, it keeps diffusing aromatic vapors, and the user cannot control the diffusing time.

An aroma sprayer generally comprises a pressure can and a liquefied aromatic substance contained in the pressure can. The user can apply a pressure to the pressure can directly or through a mechanical device, driving the pressure can to spray a fine mist of liquefied aromatic substance through a spray nozzle. This design of aroma sprayer is still not satisfactory in function. During application, the user cannot control the applied pressure to control the spray amount or spray time.

Therefore, conventional aromatic nebulizing diffusers and aroma sprayers have drawbacks as follows:
1. Not applicable to spray automatically and quantitatively at predetermined time points.
2. Not applicable to control the spray time.
3. Not applicable to control the spray amount, shortening the application time.
4. Short aroma validity.

Further, aromatic nebulizing diffusers and aroma sprayers commonly use plastic containers to hold essential oil or liquefied aromatic substances. In consequence, the essential oil or liquefied aromatic substances can easily deteriorate. Using a glass bottle or glass container can keep essential oil or liquefied aromatic substances fresh for long. However, due to manufacturing tolerance, the mounted nozzle tip may be not accurately kept in vertical at the top side of the glass bottle or glass container. After connection of a sprayer head to the nozzle tip at the top side of the glass bottle or glass container, pressing the sprayer head may be unable to cause the nozzle tip to discharge a fine mist of pressured liquefied aromatic substance.

Therefore, it is desirable to provide an automatic aroma sprayer, which eliminates the aforesaid drawbacks.

### SUMMARY OF THE INVENTION

The present invention has been accomplished under the circumstances in view. It is main object of the present invention to provide an automatic aroma sprayer, which automatically sprays a predetermined amount of fine mist of aromatic fluid droplets at a predetermined time interval.

To achieve this and other objects of the present invention, an automatic aroma sprayer in accordance with the present invention comprises a housing, which comprises an accommodation chamber, a retaining chamber disposed adjacent to the accommodation chamber and adapted for accommodating an aroma bottle, and a spray hole unit in communication between the accommodation chamber and the space outside the housing, a lower gearbox shell mounted in the accommodation chamber inside the housing and defining a hole therein, an upper gearbox shell mounted in the accommodation chamber inside the housing and facing toward the lower gearbox shell and defining a sliding guideway and a constraint slot, a pressure bar partially inserted into and axially movable along the sliding guideway and defining therein a toothed slot, a motor having the motor shaft thereof inserted through the hole of the lower gearbox shell, a gear set meshed with the toothed slot and rotatable by the motor shaft of the motor to move the pressure bar vertically up and down along the sliding guideway of the upper gearbox shell, a sprayer head stopped against the pressure bar and having a spray nozzle thereof inserted through the constraint slot and the spray hole unit of the housing, and an aroma bottle mounted in the retaining chamber of said housing and having a top nozzle tip thereof connected to the sprayer head.

The automatic aroma sprayer further comprises a functional circuit board electrically connected to the motor for controlling the motor to operate at predetermined time points, and a power circuit board electrically connected to the functional circuit board to provide the functional circuit board and the motor with the necessary working power supply.

Further, the aroma bottle uses a glass bottle holding a liquefied aromatic substance and filled up with a compressed gas. The retaining chamber of the housing is so configured that the aroma bottle can be quickly and accurately mounted in the retaining chamber to let the top nozzle tip thereof be accurately connected to the sprayer head for discharging a predetermined amount of a fine mist of liquefied aromatic substance through the spray nozzle into the outside open air.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an elevational view of an automatic aroma sprayer in accordance with the present invention.

FIG. 2 is an exploded view of the automatic aroma sprayer in accordance with the present invention.

FIG. 3 is a schematic drawing of the present invention, illustrating the bottle sleeve, the aroma bottle and the bottom cap assembled.

FIG. 4 is a sectional view of the automatic aroma sprayer in accordance with the present invention.

FIG. 5 is a schematic bottom view of the present invention, illustrating the bottom cover rotatably detachably coupled to the bottom side of the housing.

### DETAILED DESCRIPTION OF THE PREFERRED EMBODIMENT

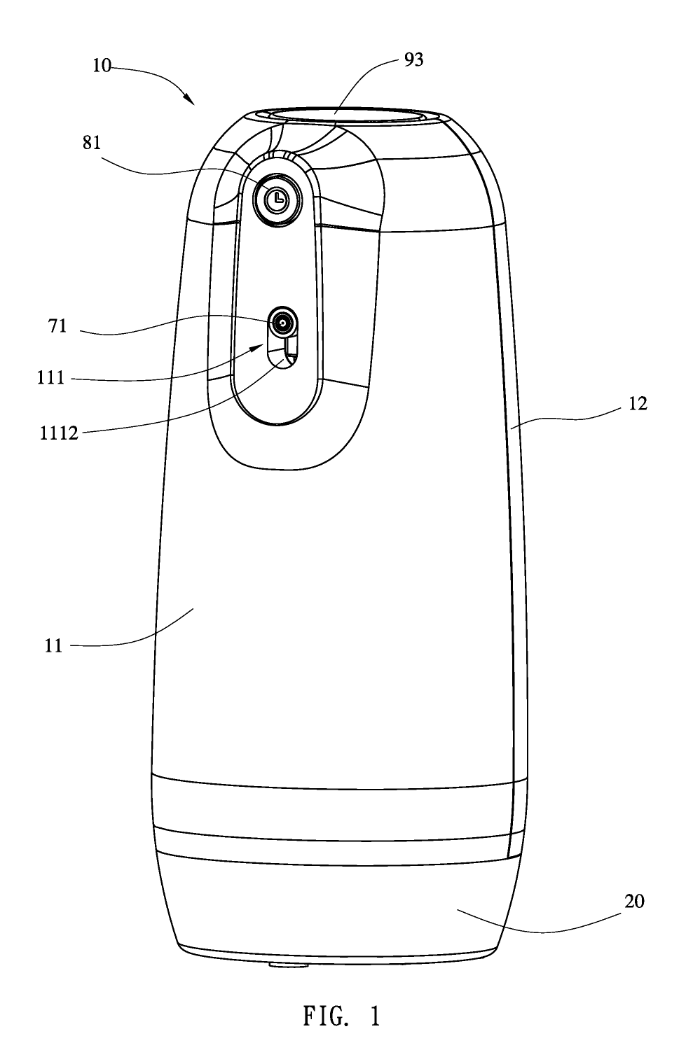

Referring to FIGS. 1, 2 and 4, an automatic aroma sprayer in accordance with the present invention is shown. The aroma sprayer comprises:

a housing 10 defining an accommodation chamber 101 therein, a retaining chamber 141 at one side thereof, and a spray hole unit 111 in communication between the accommodation chamber 101 and the space outside the housing 10;

a lower gearbox shell 30 mounted in the accommodation chamber 101 inside the housing 10 and defining a hole 301 therein;

an upper gearbox shell 40 mounted in the accommodation chamber 101 inside the housing 10 and facing toward the lower gearbox shell 30 and defining a sliding guideway 41 and a constraint slot 42;

a pressure bar 50 partially inserted into and axially movable along the sliding guideway 41 and defining therein a toothed slot 51;

a motor 60 comprising a motor shaft 61 inserted through the hole 301 of the lower gearbox shell 30;

a gear set 33 meshed with the toothed slot 51 and rotatable by the motor shaft 61 of the motor 60 to move the pressure bar 50 upward or downward along the sliding guideway 41 of the upper gearbox shell 40;

a sprayer head 70 stopped against the pressure bar 50 and having a spray nozzle 71 thereof inserted through the constraint slot 42 and the spray hole unit 111 of the housing 10; and

an aroma bottle 72 mounted in the retaining chamber 141 and having a top nozzle tip 721 thereof connected to the spray nozzle 71 of the sprayer head 70.

When the motor 60 is electrically conducted, the motor 60 drives the gear set 33 to move the pressure bar 50 downwardly along the sliding guideway 41 of the upper gearbox shell 40, forcing the spray nozzle 71 of the sprayer head 70 against the top nozzle tip 721 of the aroma bottle 72, and therefore a fine spray of liquefied aromatic substance is sprayed out of the aroma bottle 72 through the top nozzle tip 721 and the spray nozzle 71 into the outside open air.

The automatic aroma sprayer further comprises a functional circuit board 80 electrically connected to the motor 60 and adapted for controlling the motor 60 to work at predetermined time interval within a predetermined time period, and a power circuit board 9 electrically connected to the functional circuit board 80 to provide the necessary working power supply. Thus, the sprayer head 70 can spray a predetermined amount of a fine spray of liquefied aromatic substance automatically at a predetermined time interval.

Further, after the predetermined time period is up, the pressure applied to the pressure bar 50 disappears. At this time, the top nozzle tip 721 of the aroma bottle 72 returns to its former position, thereby returning the pressure bar 50 and the spray nozzle 71 of the sprayer head 70 to their former positions for a next operating cycle.

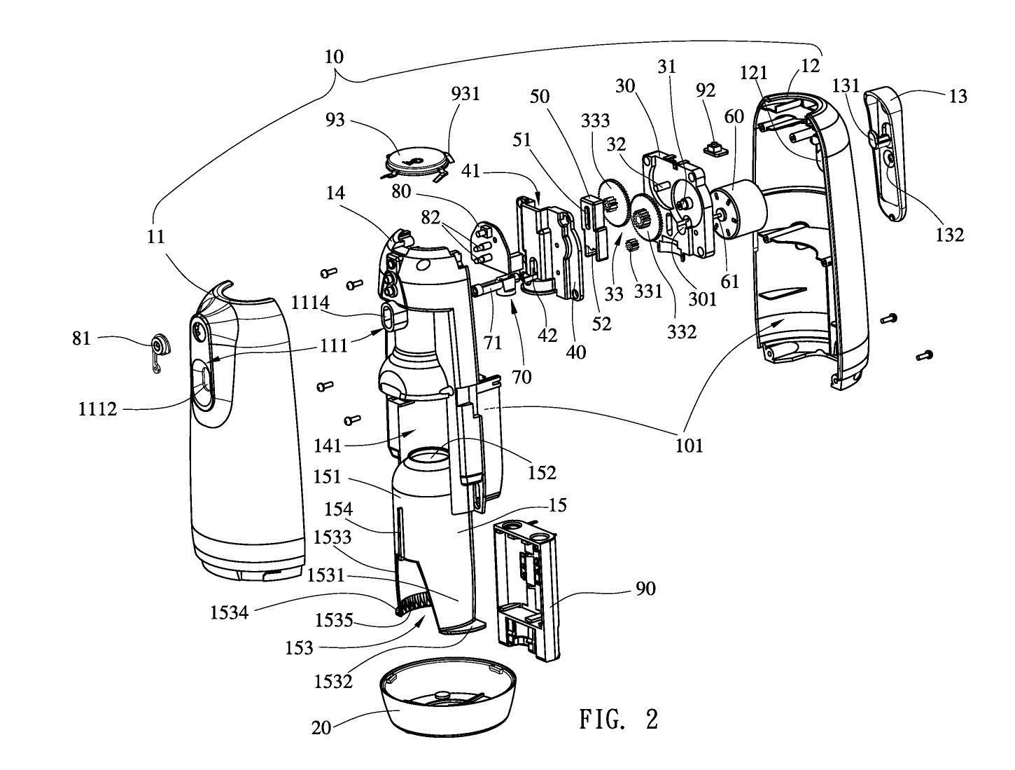

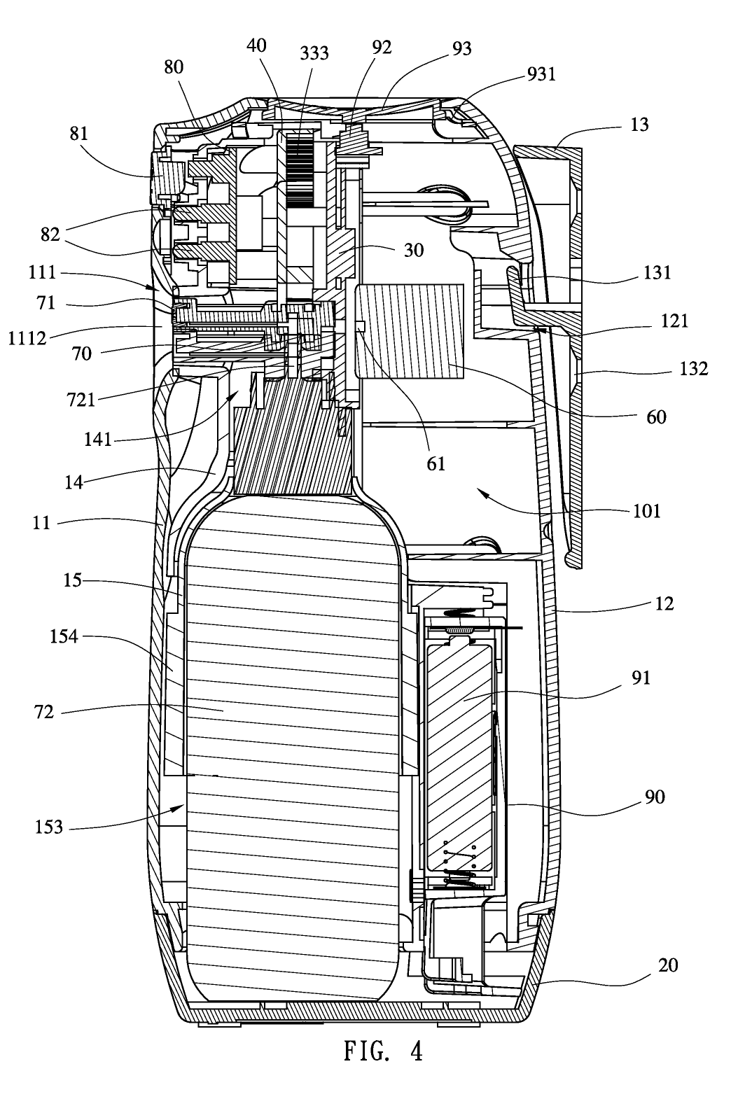

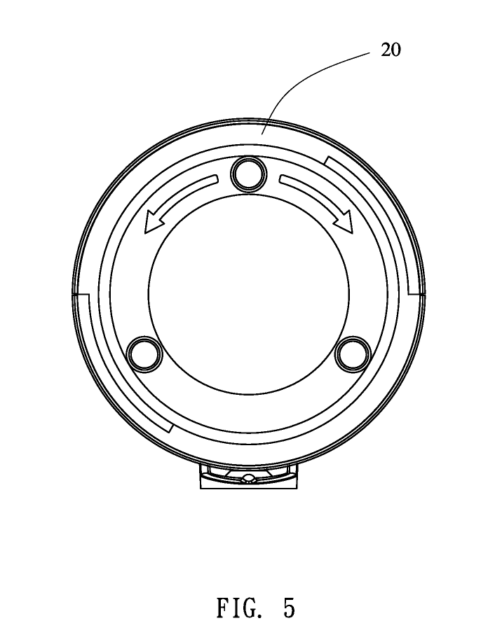

Referring to FIGS. 2 and 4 again, the automatic aroma sprayer further comprises a bottom cap 20 coupled to the bottom side of the housing 10. Referring also to FIG. 5, a rotary coupling structure is provided between the bottom side of the housing 10 and the top side of the bottom cap 20 so that the bottom cap 20 can be locked to the housing 10 after having been attached to the housing 10 and rotated in one direction relative to the housing 10, or detached from the housing 10 when rotated in the reversed direction.

Further, the gear set 33 comprises a first gear 331, a second gear 332 and a third gear 333. The first gear 331 can be a pinion connected to the motor shaft 61 of the motor 60. The second gear 332 is meshed between the first gear 331 and the third gear 333. The third gear 333 is meshed in the toothed slot 51, and rotatable to move the pressure bar 50 downwardly against the sprayer head 70. Further, the gear set 62 can be configured subject to the desired speed ratio, and can be coupled to the toothed slot 51 either directly or indirectly, enabling the gear set 62 to be driv3en by the motor shaft 61 to move pressure bar 50 downwardly in pressing the sprayer head 70, or upwardly away from the sprayer head 70.

Further, because the spray nozzle 71 of the sprayer head 70 is inserted through the constraint slot 42 and the spray hole unit 111 of the housing 10, it can simply be moved up and down along the constraint slot 42, avoiding horizontal displacement. Further, the pressure bar 50 has a bottom notch 52 attachable to the sprayer head 70 for pressing the sprayer head 70. As stated above, the aroma bottle 72 is mounted in the retaining chamber 141, allowing the top nozzle tip 721 to be accurately connected to the spray nozzle 71. Further, the power supply unit 90 is electrically connected to the motor 60 and the functional circuit board 80 to provide the motor 60 and the functional circuit board 80 with the necessary working power supply. According to the present preferred embodiment, the power supply unit 90 is a battery box holding a set of battery cells 91.

The functional circuit board 80 has built therein a control circuit (not shown) for controlling the motor 60 to work at a predetermined time interval. When the motor 60 is electrically conducted to rotate the motor shaft 61, the motor shaft 61 drives the gear set 62 to move the pressure bar 50 downwardly along the sliding guideway 41 of the upper gearbox shell 40 toward the sprayer head 70, thereby pressing the sprayer head 70 to spray a predetermined amount of a fine spray of liquefied aromatic substance.

Referring to FIG. 2 again, the lower gearbox shell 30 comprises a first axle 31 and a second axle 32. The second gear 332 is rotatably supported on the first axle 31. The third gear 333 is rotatably supported on the second axle 32. Thus, the gear set 33 is rotatable to move the toothed slot 51 upward or downward.

Referring to FIGS. 1 and 2 again, the housing 10 comprises a front cover shell 11, a back cover shell 12 and an intermediate cover shell 14. The spray hole unit 111 comprises a sprayer head hole 1012 located on the front cover shell 11, and a guide hole 1014 located on the intermediate cover shell 14. The front cover shell 11 is fastened to the intermediate cover shell 14 to keep the guide hole 1014 in communication with the sprayer head hole 1012. The intermediate cover shell 14 is fastened to the back cover shell 12, defining the accommodation chamber 101 in between the back cover shell 12 and the intermediate cover shell 14. According to the present preferred embodiment, the retaining chamber 141 is a semi-arched chamber fitting the cylindrical outer perimeter of the aroma bottle 72. The retaining chamber 141 is defined in the bottom side of the intermediate cover shell 14.

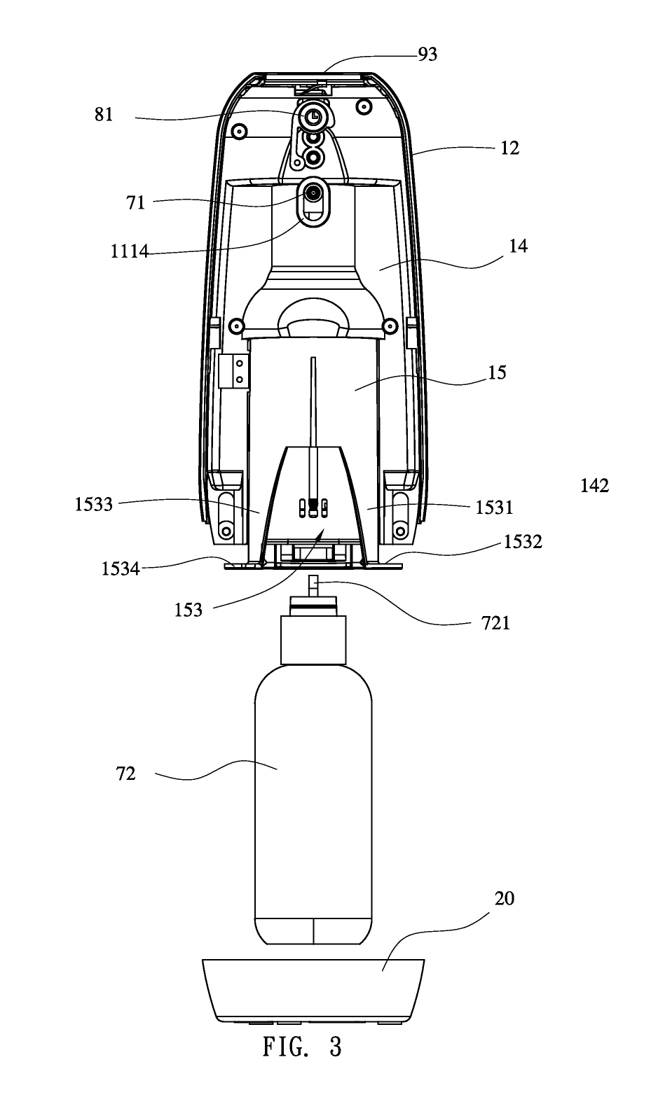

Referring to FIG. 3, the aforesaid aroma bottle 72 is a glass bottle holding therein a liquefied aromatic substance and filled up with a compressed gas. The glass material property enables the contained liquefied aromatic substance to be kept fresh for a long period of time. Instead of the use of a plastic aroma bottle in conventional designs, the invention allows the top nozzle tip 721 of the glass aroma bottle 72 to be accurately and positively connected to the sprayer head 70 so that the sprayer head 70 can be accurately and positively pressed by the bottom notch 52 of the pressure bar 50, causing the aroma bottle 72 to discharge a predetermined amount of a fine spray of liquefied aromatic substance.

Referring to FIGS. 2 and 3 again, a bottle sleeve 15 is mounted in the retaining chamber 141 of the housing 10 for holding the aroma bottle 72. The bottle sleeve 15 is configured subject to the configuration of the retaining chamber 141. Further, the bottle sleeve 15 is detachably mounted in the retaining chamber 141 for holding the aroma bottle 72.

The bottle sleeve 15 comprises a sleeve body 151, a top neck 152 located at one side, namely, the top side of the sleeve body 151, at least one, for example, two longitudinal slits 154 formed in the sleeve body 151, a first clamping plate 1531 and a second clamping plate 1533 bilaterally downwardly extended from an opposite side, namely, the bottom side of the sleeve body 151 and separated by the longitudinal slits 154, and a bottom opening 153 defined at the bottom side of the sleeve body 151 and surrounded by the first clamping plate 1531 and the second clamping plate 1533. The longitudinal slits 154 make the first clamping plate 1531 and the second clamping plate 1533 elastically deformable so that the aroma bottle 72 can be conveniently and upwardly inserted through the bottom opening 153 into the inside of the sleeve body 151 and then clamped in place by the first clamping plate 1531 and the second clamping plate 1533. After installation of the aroma bottle 72 in the bottle sleeve 15, the top nozzle tip 721 is extended out of the top neck 152 of the bottle sleeve 15 and accurately and positively connected to the sprayer head 70.

Further, the first clamping plate 1531 has its bottom side terminating in an outwardly extended horizontal stop flange 1532; the second clamping plate 1533 has its bottom side terminating in an outwardly extended horizontal stop flange 1534. After installation of the bottle sleeve 15 in the retaining chamber 141, the stop flange 1532 of the first clamping plate 1531 and the stop flange 1534 of the second clamping plate 1533 are respectively stopped at the bottom side of the intermediate cover shell 14. After installation of the hard glass aroma bottle 72 in the bottle sleeve 15 inside the retaining chamber 141 of the intermediate cover shell 14, the aroma bottle 72 is positively secured in position. Further, by means of pulling the stop flange 1532 of the first clamping plate 1531 and the stop flange 1534 of the second clamping plate 1533, the bottle sleeve 15 and the aroma bottle 72 can easily and quickly be removed from the retaining chamber 141 of the intermediate cover shell 14.

Further, the bottle sleeve 15 comprises barbed friction portions 153 on the inside wall thereof for friction engagement with the inserted aroma bottle 72 to enhance connection stability between the bottle sleeve 15 and the aroma bottle 72, enabling the top nozzle tip 721 of the aroma bottle 72 to be accurately and positively connected to the sprayer head 70.

Further, after removal of the bottom cap 20 from the housing 10, the user can insert the bottle sleeve 15 and the attached aroma bottle 72 into the retaining chamber 141 of the intermediate cover shell 14 and connect the top nozzle tip 721 of the aroma bottle 72 to the sprayer head 70 accurately.

Subject to the effect of the first clamping plate 1531 and the second clamping plate 1533, the aroma bottle 72 can be positively mounted in the bottle sleeve 15 and then mounted with the bottle sleeve 15 in the retaining chamber 141 of the intermediate cover shell 14, letting the top nozzle tip 721 of the aroma bottle 72 be accurately connected to the sprayer head 70. Further, after the aroma bottle 72 is used up, the user can pull the stop flange 1532 of the first clamping plate 1531 and the stop flange 1534 of the second clamping plate 1533 to remove the bottle sleeve 15 and the aroma bottle 72 out of the retaining chamber 141 of the intermediate cover shell 14 for a replacement.

Referring to FIGS. 2 and 4 again, the automatic aroma sprayer further comprises a mode control button 81 disposed on the outside of the housing 10 and electrically connected to the functional circuit board 80. The mode control button 81 is operable to control the functional circuit board 80, selecting one of a series of spray modes.

The automatic aroma sprayer further comprises a power button 93 electrically connected to the power supply unit 90, the power circuit board 92 and the functional circuit board 80. The power button 93 is operable to turn on/off the power circuit board 92, thereby controlling the supply of power supply from the power supply unit 90 to the functional circuit board 80 and the motor 60. The power circuit board 92 comprises a key switch (not shown) stopped at the bottom side of the power button 93. The user can operate the power button 93 to switch on/off the key switch of the power circuit board 92, thereby turning on/off the power circuit board 92.

The automatic aroma sprayer further comprises a wall hook 13. The wall hook 13 comprises at least one mounting hole 132 mountable to a support wall by at least one fastener (not shown), and a hook 131. The back cover shell 12 of the housing 10 has a hanging hole 121 for hanging on the hook 131 of the wall hook 13. Thus, by means of fastening the wall hook 13 to a support wall, the housing 10 can be hung on the wall hook 13 at the support wall.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. An automatic aroma sprayer, comprising:
a housing comprising an accommodation chamber, a retaining chamber disposed adjacent to said accommodation chamber and adapted for accommodating an aroma bottle, and a spray hole unit in communication between said accommodation chamber and the space outside said housing;
a lower gearbox shell mounted in said accommodation chamber inside said housing, said lower gearbox shell defining a hole therein;
an upper gearbox shell mounted in said accommodation chamber inside said housing and facing toward said lower gearbox shell, said upper gearbox shell defining a sliding guideway and a constraint slot;
a pressure bar partially inserted into and axially movable along said sliding guideway, said pressure bar defining therein a toothed slot;
a motor comprising a motor shaft inserted through the hole of said lower gearbox shell;
a gear set meshed with said toothed slot and rotatable by said motor shaft of said motor to move said pressure bar vertically up and down along said sliding guideway of said upper gearbox shell; and
a sprayer head stopped against said pressure bar, said sprayer head comprising a spray nozzle inserted through said constraint slot and said spray hole unit of said housing.

2. The automatic aroma sprayer as claimed in claim 1, further comprising a functional circuit board electrically connected to said motor for controlling said motor to operate at predetermined time points, and a power circuit board electrically connected to said functional circuit board to provide said functional circuit board and said motor with the necessary working power supply.

3. The automatic aroma sprayer as claimed in claim 1, wherein said gear set comprises a first gear connected to said motor shaft of said motor and rotatable by said motor shaft of said motor, a second gear meshed with said first gear, and a third gear meshed in said toothed slot and rotatable by said second gear to move said pressure bar.

4. The automatic aroma sprayer as claimed in claim 1, wherein said pressure bar comprises a bottom notch attachable to said sprayer head for pressing said sprayer head.

5. The automatic aroma sprayer as claimed in claim 3, wherein said pressure bar comprises a bottom notch attachable to said sprayer head for pressing said sprayer head.

6. The automatic aroma sprayer as claimed in claim 1, wherein said housing comprises a front cover shell, a back cover shell connected to said front cover shell, and an intermediate cover shell mounted in between said front cover shell and said back cover shell; said spray hole unit comprises a sprayer head hole located on said front cover shell, and a guide hole located on said intermediate cover shell in communication with said sprayer head hole; said accommodation chamber is defined in between said back cover shell and said intermediate cover shell.

7. The automatic aroma sprayer as claimed in claim 6, wherein said retaining chamber is defined in a bottom side of said intermediate cover shell.

8. The automatic aroma sprayer as claimed in claim 1, further comprising a bottle sleeve mounted in said retaining chamber of said housing and adapted to hold an aroma bottle, said bottle sleeve being configured subject to the configuration of said retaining chamber.

9. The automatic aroma sprayer as claimed in claim 7, further comprising a bottle sleeve mounted in said retaining chamber of said housing and adapted to hold an aroma bottle, said bottle sleeve being configured subject to the configuration of said retaining chamber.

10. The automatic aroma sprayer as claimed in claim 8, wherein said bottle sleeve comprises a sleeve body, a top neck located at a top side of said sleeve body, a plurality of clamping plates downwardly extended from a bottom side of said sleeve body, a bottom opening defined in a bottom side of said sleeve body and surrounded by said clamping plates, and a plurality of longitudinal slit formed in said sleeve body between each two adjacent said clamping plates.

11. The automatic aroma sprayer as claimed in claim 10, wherein each said clamping plate has a bottom side thereof terminating in an outwardly extended horizontal stop flange and stoppable at a bottom side of said intermediate cover shell.

12. The automatic aroma sprayer as claimed in claim 10, wherein said bottle sleeve further comprises a plurality of barbed friction portions located at an inner bottom side of said sleeve body for friction engagement with an inserted aroma bottle.

13. The automatic aroma sprayer as claimed in claim 2, further comprising a power button and a power supply unit, said power circuit board being electrically connected to said power supply unit and said functional circuit board, said power button being adapted to switch on/off said power circuit board in controlling the supply of power supply from said power supply unit to said functional circuit board and said motor.

14. The automatic aroma sprayer as claimed in claim 2, further comprising a mode control button mounted outside said housing and electrically connected to said functional circuit board for selectively controlling said functional circuit board to perform one of a series of spray modes.

15. The automatic aroma sprayer as claimed in claim 1, further comprising a wall mounting structure, said wall mounting structure comprising a wall hook for fastening to a support wall, and a hanging hole located at said back cover shell for hanging on said wall hook.

16. The automatic aroma sprayer as claimed in claim 1, further comprising a bottom cap detachably capped on said housing at a bottom side.
